(19) United States
(12) Patent Application Publication
(10) Pub. No.: US 2023/0190234 A1
(43) Pub. Date: Jun. 22, 2023

(54) ACOUSTIC HEADLIGHT

(71) Applicant: SmartCone Technologies, Inc., Arnprior (CA)

(72) Inventors: Jacques Guigné, Paradise (CA); Robert Lilly, Paradise (CA)

(21) Appl. No.: 17/930,574

(22) Filed: Sep. 8, 2022

Related U.S. Application Data
(63) Continuation of application No. PCT/IB2021/052004, filed on Mar. 10, 2021.
(60) Provisional application No. 62/987,844, filed on Mar. 10, 2020.

(51) Int. Cl. A61B 8/00

(52) U.S. Cl. CPC A61B 8/4483; A61B 8/4272; A61B 2562/0204

(57) ABSTRACT

A method for acoustic imaging includes emitting an acoustic signal. The method includes (a) emitting an acoustic signal; (b) detecting signals corresponding to reflections of the acoustic signal at an array of positions proximate a positon of the emitting; (c) adding, for a chosen distance from the position of the emitting, a respective time delay to each detected signal to cause each detected signal to correspond to each of a plurality of positions in a plane transverse to a line normal to a plane of the array of positions; (d) summing the time delayed signals; (e) selecting one of the plurality of positions having a highest summed, delayed signal amplitude; and (f) assigning a pixel to the one of the plurality of positions having the highest amplitude. (b) through (f) are repeated for a plurality of different chosen distances.

### CROSS REFERENCE TO RELATED APPLICATIONS

Continuation of International Application No. PCT/IB2021/052004 filed on Mar. 10, 2021. Priority is claimed from U.S. Provisional Application No. 62/987,844 filed on Mar. 10, 2020. Both the foregoing applications are incoroporated herein by reference in their entirety.

### STATEMENT REGARDING FEDERALLY SPONSORED RESEARCH OR DEVELOPMENT

Not Applicable

### NAMES OF THE PARTIES TO A JOINT RESEARCH AGREEMENT

Not Applicable.

### BACKGROUND

This disclosure relates to the field of hazard detection for motor vehicles. More specifically, the disclosure relates to acoustic-based sensors for locating objects presenting a hazard to vehicular movement.

The detection of people, objects or other vehicles in the forward or reversing path of potential vehicle movement is of extreme importance. Systems known in the art for detecting such objects, other than visual detection, include radar-based and acoustic-based parking sensors. Such sensors may crudely determine approximate range to an object in the path of the vehicle, but lack resolution to determine whether an obstacle exists in the path of a rapidly moving vehicle, and sufficient range to alert a vehicle operator in time to be useful when a vehicle is moving quickly.

### SUMMARY

One aspect of the present disclosure relates to a method for acoustic imaging. A method according to this aspect of the disclosure includes emitting an acoustic signal. The method includes (a) emitting an acoustic signal; (b) detecting signals corresponding to reflections of the acoustic signal at an array of positions proximate a positon of the emitting; (c) adding, for a chosen distance from the position of the emitting, a respective time delay to each detected signal to cause each detected signal to correspond to each of a plurality of positions in a plane transverse to a line normal to a plane of the array of positions; (d) summing the time delayed signals; (e) selecting one of the plurality of positions having a highest summed, delayed signal amplitude; and (f) assigning a pixel to the one of the plurality of positions having the highest amplitude. (b) through (f) are repeated for a plurality of different chosen distances.

In some embodiments, the emitted acoustic signal comprises a coded signal.

Some embodiments further comprise cross-correlating each detected signal with the coded emitted signal to determine a two way travel time of the emitted signal to be reflected from an object.

In some embodiments, the emitting an acoustic signal is performed at two different frequencies.

In some embodiments, the two different frequencies are 25 kHz and 40 kHz.

In some embodiments, the plurality of positions is disposed in a ring about the position of emitting.

In some embodiments, the plurality of positions is disposed in two, crossed lines passing through the position of emitting.

An acoustic headlamp according to another aspect of this disclosure includes optical lamp housing, an acoustic transmitter disposed on the optical lamp housing, a plurality of acoustic receivers disposed in a selected pattern about the optical lamp housing, and circuitry for causing the transmitter to emit pulses of acoustic energy and detecting signals generated by the plurality of receivers. The circuitry is operable to: (a) add, for a chosen distance from the transmitter, a respective time delay to each detected signal to cause each detected signal to correspond to each of a plurality of positions in a plane transverse to a line normal to a plane of the array of positions; (b) sum the time delayed signals; (c) select one of the plurality of positions having a highest summed, delayed signal amplitude; (d) assign a pixel to the one of the plurality of positions having the highest amplitude; and repeat (a) through (d) for a plurality of different chosen distances.

In some embodiments, the plurality of receivers is arranged in a circle about a perimeter of the optical lamp housing.

In some embodiments, the plurality of receivers is arranged in two orthogonal lines.

In some embodiments, the acoustic energy pulses comprise coded signals.

In some embodiments, the acoustic energy pulses comprise at least two frequencies.

In some embodiments, the at least two frequencies comprise 25 kHz and 40 kHz.

Other aspects and possible advantages will be apparent from the description and claims that follow.

### BRIEF DESCRIPTION OF THE DRAWINGS

FIGS. 1A, 1B and 1C show example arrangements of an acoustic transmitter and arrays of acoustic receivers in accordance with the present disclosure.

FIG. 2 graphically shows geometry of acoustic signal emission and detection with respect to objects in the imaging field.

FIG. 3 is a flow chart of an example embodiment of imaging.

### DETAILED DESCRIPTION

An apparatus according to the present disclosure may produce sensing from an acoustics-based “headlamp” styled detection platform. An example embodiment may use transducers operating within acoustic frequencies between 20 kHz and 40 kHz. A form-fitted, automotive headlamp-like architecture of the apparatus may be used to support the active acoustics and beamforming protocols with an answer-product software output produced either in front of the vehicle operator or integrated directly into a vehicular decision-making set of alarms and braking controls.

An apparatus according to the disclosure may include a look-ahead warning for drivers of vehicles in visually difficult conditions such as fog, snow, or heavy rain, under which optics are less likely to provide reliable information. Signal to noise calculations suggest that 25 kHz may be useful out to approximately 17 meters range while 40 kHz may be useful at ranges less than approximately 10 meters. It is possible to obtain usable target imaging to about 100 meters at lower frequencies, e.g, 8 to 10 kHz if suitable signal processing is used to address vehicle noise in such frequency range.

The acoustic transducers, both transmitting and receiving, may be accommodated within the confines of a housing such as a vehicular headlamp housing. Certain implementations may allow the headlight to be dual purpose, in that it continues to function as an optical headlight as well as an acoustic system according to the present disclosure. Other embodiments may simply use the familiar housing of an automotive headlamp to be solely an acoustic device. One possible embodiment may comprise a transmitter and receivers arranged as will be further explained on the exterior of a parabolic aluminized reflector (PAR) lamp housing. An optical lamp may be disposed in such a PAR housing as is known in the art, for example a halogen lamp, high intensity discharge (HID) lamp or light emitting diode (LED) lamp.

The acoustic components of the apparatus may be used to detect acoustic scatter returned from objects in the path of the vehicle that may be present within an insonification volume extending along the direction of travel of the vehicle. Following an acoustic transmission, detected acoustic signals corresponding to a specific distance (range) and in a narrow receiving beam may be stored in a processor as pixels in an array corresponding to a vertical plane at a specific distance from (e.g., in the direction of motion) the vehicle. Many such planes of pixels may be stored in the processor, e.g., in a memory, with suitable distance intervals extending ahead of the vehicle position to a maximum search range related to the frequency of acoustic energy used. This allows imaging of objects in a zoom in/zoom out capaability to include analyzing the images to provide alarms of hazards.

The acoustic transmissions may be centered on two frequencies: a lower frequency, for example, 25 kHz, would enable acceptable resolution to capture echo images out to 20 meters; a higher frequency, for example, 40 kHz, would enable higher resolution images to be obtained at ranges out to 10 meters. These two types of images may be used to complement each other in image processing. A vehicle moving at 50 km/hour corresponds to 14 m/s. The update rate of the images depends the repetition rate of the transmission which is determined by the maximum range (17 meters range corresponds to two-way acoustic travel time of about 0.1 seconds) and the processing time. So when looking “ahead” using 25 kHz acoustic energy and a hazard is detected at 15 meters, by the time the image is refreshed, for example, each ¼ second, the vehicle has moved 3.5 meters. A second refresh brings the hazard to 8 meters ahead, well within the range of the 40 kHz acoustic signal, which has finer resolution than the 25 kHz signal. However, an update rate approaching the repetition rate of the transmission may be possible with optimized processing software, e.g., 10 times per second. The foregoing frequency and range parameters are intended as examples and are not intended to limit the scope of the present disclosure.

An effective short duration acoustic pulse may be transmitted which provides insonification in a spherical “shell” whose thickness is determined by the duration of the pulse and an angular extent of the shell determined by the design of the transducer. Objects within the shell scatter the incident signal, and some of the incident signal is returned to the receivers in the apparatus.

The acoustic transmitter is preferably broad beam (large beam subtended angle) in order to insonify a substantial volume ahead of the vehicle. The width of a radiated acoustic beam depends on the size of the radiating aperture in wavelengths. For example, for a circular plane source of diameter 1 wavelength (of the emitted acoustic energy), the main lobe of the beam is approximately 60 degrees subtended angle. This means that linear cross section dimensions of the spherical shell, of thickness dependent on effective pulse length, at a particular range is equal to the range. Increasing the coverage requires increasing the acoustic propagating beam. Decreasing the size of the transmitter in terms of wavelength would increase the beam width but significantly reduce the amount of energy that can be radiated. By configuring the transmitter physical embodiment with a spherically shaped cap over the active face of the transmitter, both the beam width can be increased and the radiating surface may deliver a wide covering transmitting beam with sufficient signal strength. In some embodiments, several acoustic transmitters could be used, each of main beam 60 degrees subtended angle with their respective beam axes splayed to obtain the coverage required. Each transmitter in such a case would need its transmitted signal coded such that on reception, detected signals can be correctly assigned to the respective transmitter.

Emitting an effective short acoustic pulse is useful for image range resolution. Transmission of a short time duration acoustic pulse, however, limits the energy that may be transmitted as a result of large peak values of voltage/current imparted to the transducer in a short time duration pulse. By transmitting a long time duration signal of duration t with its energy distributed over a relatively large bandwidth BW, an equivalent to a short time duration pulse may result from a cross correlation of the received signals with the (“seed” or waveform of) transmitted signal, with a large peak amplitude thus avoided. The transmitted signal can be, for example, any pseudo-random coded signal with a known “seed.”

Combining the received signal from each of the receiving sensors can be performed in the processor and thus used to create a narrow effective receiving beam directed in any desired direction, e.g., by application of suitable time delays to respective receiver signals. Thus, the position of an object creating an acoustic echo can be located in three dimensions within the illuminating shell of the transmitter: the range may be obtained from the elapsed time from transmission, and two angles may be determined from reception beamforming.

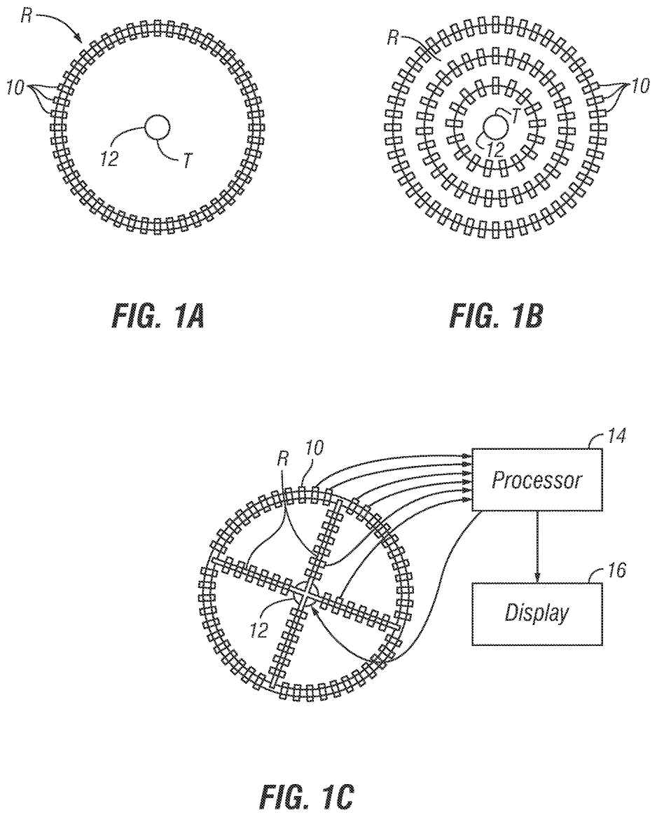

FIGS. 1A, 1B and 1C shows possible arrangements for acoustic components according to the present disclosure. In FIG. 1A, a transmitter 12 may be disposed within receivers 10 may be disposed in a ring R as shown in the figure. The tramsitter 12 and receivers 10 may be in signal communication with a processor 14. The processor 14 may be any form of computer, microcomputer, field programmable gate array, programmable logic controller or the like. The illustrated arrangement of receivers 10 may produce a narrow receiver beam with side lobes about 8 dB below a main beam. In FIG. 1B, filled aperture receivers 10 may be disposed throughout the area defined by the ring R to produce a narrow beam of the same angular width as the ring array in FIG. 1A, but the side lobes of the receiver beam are about 12 dB below the main beam.

The diameter of the ring R in FIG. 1A may be selected, for example, to fit around the periphery of a standard automotive headlamp, and for example purposes as about 30 cm diameter. The near field of the receiver array extends to about 10 m at 40 kHz and to about 7 m at 25 kHz, given the receiving ring array R diameter of 30 cm. So beamforming in the present context is near field beamforming. That is, the far field beam pattern is reproduced in the near field, provided the beamforming recognizes that scatterers will be at finite distances.

The far field beam width of the receiving beam in the receiver arrangement of FIG. 1A is determined by the diameter of the ring R of receivers 10. For example, for a ring R diameter of 30 cm and an acoustic frequency of 40 kHz (wavelength 0.9 cm) the far field −3 dB beam width of the main lobe would be 1.7 degrees, requiring 220 half-wavelength spaced receivers 10 around the ring R. If the transmitter beam subtends an angle of 60 degrees, then a beamformed receiver would supply an image of reflections (echoes) received from each particular range with a minimum number of independent pixels equal to the number of receivers. For a lower frequency, e.g., 25 kHz, with a far field −3 dB main lobe of about 2.7 degrees, the number of half wavelength spaced receivers would be 140. The image quality will be greater for greater values of directivity index.

Side lobes can be a problem when the main beam is directed toward a low strength scatterer while the side lobes are directed to a high strength scatterer. Further, the ability to reject ambient noise depends on the number of receivers used to beam form, which effectively combines the main beam with and the side lobe levels. This noise rejecting performance is expressed by the parameter called directivity index (DI). For a ring array of diameter 30 cm, at an acoustic frequency of 40 kHz, the DI is 23.4 dB, (for the case of a plane circular array at 40 kHz the DI=40.8 dB). At a frequency of 25 kHz, a ring array diameter of 30 cm results in the DI being 21.4 dB.

If noise is a problem for a ring array as in FIG. 1A, the DI can be increased by including some radial line arrays of receivers 10, as shown at R1 in FIG. 1C. Using line arrays R1 as in FIG. 1C would increase signal processing complexity, however.

The number of receivers 10 may be chosen such that the spacing between them is one-half wavelength of the acoustic energy used. This spacing would enable steering beamforming over an angular range of +/−90 degrees from a line perpendicular to the plane of the ring array R. To accommodate the steering requirements of several transmitters as described above the receiver spacing will be taken as a half wavelength.

The transmitter 12 and receivers 10 may be connected to circuitry, e.g., the processor 14 that can actuate the transmitter 12, and detect signals generated by the receivers 10 in response to detected acoustic energy. The processor 14 may comprise signal generating and processing functionality to be explained further below. The processor 14 may be connected to a display 16 for use by the vehicle operator, or the processor 14 may be in signal communication with existing vehicle electronic devices to provide a visual display or other signal device for the vehicle operator.

For 40 kHz acoustic frequency and a ring receiver array of 30 cm diameter, the number of receiving sensors required is about 220 to obtain the above described half wavelength spacing. If the frequency is 25 kHz, for the same ring array diameter the number of receiver needed is about 140, as explained above. Receiving sensors sufficiently small to enable half wavelength spacing at the frequencies described herein (40 kHz or 25 kHz) may be in the form of Micro-Electro-Mechanical systems (MEMs) transducers, or more specifically, capacitive MEMs ultrasonic transducers (CMUTs) which have been developed as an alternative form of transducer offering advantages such as wide bandwidth, ease of fabricating large arrays, and potential for integration with electronics. The foregoing technology makes it possible to create large arrays using simple photolithography. Two-dimensional CMUT arrays with as many as 128×128 elements have already been successfully fabricated and characterized.

Individual electrical connections to transducer elements may be provided by through-wafer interconnects. MEMs ultrasonic transducers are commercially available e.g., model number SPM0204 sold by Knowles Electronics, LLC. Itasca, Ill. Such transducers provide usable sensitivity as well as a nearly flat response between 10 kHz and 70 kHz. Their size, 4.72×3.76×1.15 mm, makes them useful for compact ultrasonic applications.

An example embodiment to be made to fit within a single headlight type enclosure of diameter 30 cm may comprise the following. A transmitter, with a 60 degree subtended angle beam width and designed to emit both 25 kHz and 40 kHz energy with a bandwidth of up to 5 kHz may be used. For greater coverage there may be more than one transmitter, with axes splayed. The transmitter (or transmittes) may be disposed in the center of a receiver array as explained with reference to FIGS. 1A, 1B and 1C. Around the circumference shown in FIG. 1A, receiving sensors such as a CMUT array as explained above may deployed at half wavelength spacing between individual receiver elementws. There may be, for a 40 kHz operating frequency, 220 receiving sensors (10 in FIG. 1A) in such an array. The transmitter (12 in FIG. 1A) may be designed to be centered both on 40 kHz and 25 kHz. The same receivers can be used at both frequencies. The transmitter and receiver bandwidths should be such that suitably short acoustic pulses can effectively be transmitted.

A pseudo random coded signal may be transmitted by the transmitter as explained above. If more than one transmitter is used, each transmitter may have a different cide (seed) for such signal to enable signal detection that can be related to the specific transmitter signals. Acoustic signal transmission centred on both 40 kHz and 25 kHz can be simultaneously emitted, again, using unique coding for each of the 25 kHz and 40 kHz signals.

The received signal detected by each receiving sensor may be obtained in time windows having duration equivalent to the two way acoustic travel time to the maximum range of the headlight. These signals will be an additive mixture of the pseudo random noise signals returned by scattering from all scatterers as a result of radiation from the single or all transmitters. For each receiving sensor, after separation by temporal filtering of the 25 kHz and 40 kHz signals, the received signal may be cross correlated in turn with each of the different coded transmitted signals. Thus, as a result of the cross correlation, associated with each receiving sensor is a time series equivalent to what would have been detected by having transmitted a large amplitude, short duration signal from the or each of the transmitters separately, at each of two center frequencies.

A small range interval may be selected, which interval may be determined by the transmitted bandwidth, for example, about 5 kHz, at a particular range and each of the three time series from each of the receiving sensors is near-field beam formed (spatially filtered) to form narrow beams within the (60 degree angle) transmitter beam cone. Thus for each 60-degree cone illumination there are many possible peak value of each narrow beam, which can be termed “pixels.”

A divided screen on the display (e.g., 16 in FIG. 1C) may show a selected number of ranges of distance ahead of the vehicle. The image for each of many range windows can be calculated and stored for display by the user (zoom in and out) or stored by the processor (14 in FIG. 1C) for analysis of hazard detection.

Images for both the frequencies, 40 kHz and 25 kHz, can be made simultaneously available. The 40 kHz signals will provide greater angular resolutions in the range out to about 10 m, and the 25 kHz may provide a coarser angular resolution over a whole range out to about 20 m.

An update rate of the images depends the repetition rate of the transmission which is determined by the maximum range (17 m range corresponds to two way travel time of 0.1 sec) and on the processing time.

Beamforming the received signals to extract range and position information may be described as follows. The time from the instant of signal transmission to reception of a signal at position (x,y) is defined by the expression:

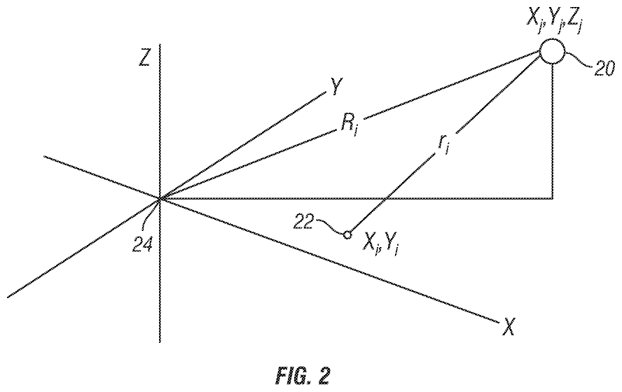

Tᵢⱼ=Rⱼ+rᵢ

where j identifies a scatterer, and i identifies the receiver. The foregoing are shown in FIGS. 2 at 20 and 22, respectively, where 24 repesents the transmitter position. FIG. 2 may be used to understand the parameters in the following equations.

Tᵢⱼ=Rⱼ(1+(1+r₀²/Rⱼ²−(2xᵢ cos θⱼ sin φⱼ/Rⱼ−2yᵢ sin θⱼ sin φⱼ/Rⱼ)^{0.5}))

Xⱼ=Rⱼ cos θⱼ sin φⱼ

Yⱼ=Rⱼ sin θⱼ sin φⱼ

Zⱼ=Rⱼ cos φⱼ.

The arrival time of a scattered signal is governed by Tᵢⱼ. Thus, for beam forming, the output of each receiver may be shifted by a time −Tᵢⱼ+Zⱼ. Signals from all receivers are then summed to provide the array output focused on a position specified by Xⱼ, Yⱼ, Zⱼ. The value of Zⱼ is determined by an increment window of range selected for examination for the presence of targets.

The near field of the receiver array extends to about 10 m at 40 kHz and to about 7 m at 25 kHz, given the example receiver ring array diameter of 30 cm. If Rⱼ>>r₀ then the far field condition applies so that:

Tᵢⱼ2Rⱼ−xᵢ cos θⱼ sin φⱼ−yᵢ sin θⱼ sin φⱼ

And if φ=0 then the signal arrives at all receiving sensors at a time 2Rⱼ.

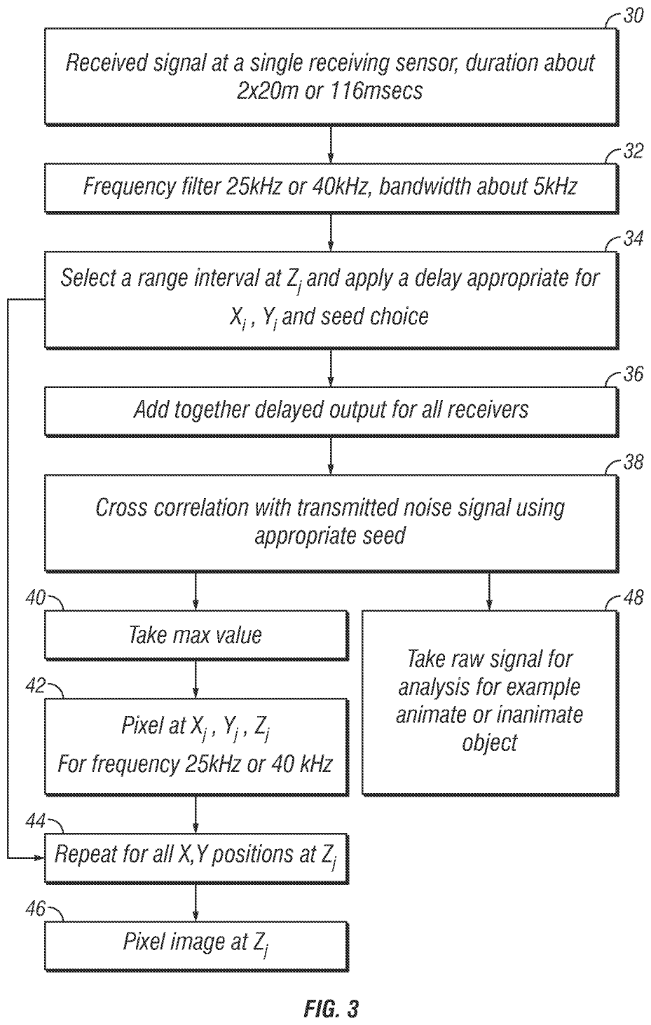

An example acquisition and processing procedure may be better understood with reference to FIG. 3. At 30, in response to actuation of the transmitter, a signal is detected at one of the receivers in the reciever array. For a 20 meter range, the total time from transmssion to reception is 116 msec. The detected signal may be filtered, at 32, by a 5 KHz passband filter first centered about 25 kHz, and then centered about 40 kHz. At 34, a range of object distance may be chosen, for which a delay time for each receiver to provide the correct distance (range) at each position Xi, Yi in the image field may be calculated and applied to the respective receiver signals. At 36, the time delayed receiver signals are summed. At 38, the summed signal is cross-correlated with the transmitter signal. At 40, a maximum value of the cross correlation provides the X, Y and Z coordinates of the location of the object in the image field. At 42, a pixel value may be determined at the X, Y, Z location. This process may be peformed for both 25 kHz and 40 kHz transmitter signals. The foregoing may be repeated for all X, Y positions at range Z. At 46, the foregoing may be repeated for other values of the range, Z.

At 48, raw receiver signal may be processed to obtain information about the type of object in the image field, such as whether it is animate (moving) or inanimate (stationary).

In light of the principles and example embodiments described and illustrated herein, it will be recognized that the example embodiments can be modified in arrangement and detail without departing from such principles. The foregoing discussion has focused on specific embodiments, but other configurations are also contemplated. In particular, even though expressions such as in “an embodiment,” or the like are used herein, these phrases are meant to generally reference embodiment possibilities, and are not intended to limit the disclosure to particular embodiment configurations. As used herein, these terms may reference the same or different embodiments that are combinable into other embodiments. As a rule, any embodiment referenced herein is freely combinable with any one or more of the other embodiments referenced herein, and any number of features of different embodiments are combinable with one another, unless indicated otherwise. Although only a few examples have been described in detail above, those skilled in the art will readily appreciate that many modifications are possible within the scope of the described examples. Accordingly, all such modifications are intended to be included within the scope of this disclosure as defined in the following claims.

What is claimed is:

1. A method for acoustic imaging, comprising:
(a) emitting an acoustic signal;
(b) detecting signals corresponding to reflections of the acoustic signal at an array of positions proximate a position of the emitting;
(c) adding, for a chosen distance from the position of the emitting, a respective time delay to each detected signal to cause each detected signal to correspond to each of a plurality of positions in a plane transverse to a line normal to a plane of the array of positions;
(d) summing the time delayed signals;
(e) selecting one of the plurality of positions having a highest summed, delayed signal amplitude;
(f) assigning a pixel to the one of the plurality of positions having the highest amplitude; and
repeating (b) through (f) for a plurality of different chosen distances.

2. The method of claim 1 wherein the emitted signal comprises a coded signal.

3. The method of claim 2 further comprising cross-correlating each detected signal with the coded emitted signal to determine a two way travel time of the emitted signal to be reflected from an object.

4. The method of claim 1 wherein the emitting an acoustic signal is performed at two different frequencies.

5. The method of claim 4 wherein the two different frequencies are 25 kHz and 40 kHz.

6. The method of claim 1 wherein the plurality of positions are disposed in a ring about the position of emitting.

7. The method of claim 1 wherein the plurality of positions are disposed in two, crossed lines passing through the position of emitting.

8. An acoustic headlamp, comprising:
an optical lamp housing;
an acoustic transmitter disposed on the optical lamp housing;
a plurality of acoustic receivers disposed in a selected pattern about the optical lamp housing; and
circuitry for causing the transmitter to emit pulses of acoustic energy, the circuitry detecting signals generated by the plurality of receivers, the circuitry operable to,
(a) add, for a chosen distance from the transmitter, a respective time delay to each detected signal to cause each detected signal to correspond to each of a plurality of positions in a plane transverse to a line normal to a plane of the array of positions;
(b) sum the time delayed signals;
(c) select one of the plurality of positions having a highest summed, delayed signal amplitude;
(d) assign a pixel to the one of the plurality of positions having the highest amplitude; and
repeat (a) through (d) for a plurality of different chosen distances.

9. The acoustic headlamp of claim 8 wherein the plurality of receivers is arranged in a circle about a perimeter of the optical lamp housing.

10. The acoustic headlamp of claim 8 wherein the plurality of receivers is arranged in two orthogonal lines.

11. The acoustic headlamp of claim 8 wherein the acoustic energy pulses comprise coded signals.

12. The acoustic headlamp of claim 8 wherein the acoustic energy pulses comprise at least two frequencies.

13. The acoustic headlamp of claim 12 wherein the at least two frequencies comprise 25 kHz and 40 kHz.
